(19) United States
(12) Patent Application Publication
(10) Pub. No.: US 2026/0325140 A1
(43) Pub. Date: Oct. 22, 2026

(54) DEVICE AND METHOD FOR OCCLUDING A TUBULAR ORGAN

(71) Applicant: FEMIGUARD LTD., Ra'anana (IL)

(72) Inventors: Shlomo Bezalel COHEN EYLON, Michmoret (IL); Tamir BEN DAVID, Tel Aviv (IL); Yitzhak OZ, Tal-El (IL)

(21) Appl. No.: 19/122,461

(22) PCT Filed: Oct. 18, 2023
(86) PCT No.: PCT/IL2023/051086

§ 371 Date: Apr. 18, 2025

Related U.S. Application Data
(60) Provisional application No. 63/380,068, filed on Oct. 19, 2022.

(51) Int. Cl. A61B 17/12; A61B 90/00

(52) U.S. Cl. CPC A61B 17/12177; A61B 17/12036; A61B 17/12104; A61B 90/39; A61B 2017/1205; A61B 2090/3937

(57) ABSTRACT

The present disclosure provides an occluding device, a method for deploying and retrieving it and a method for occluding a tubular organ. The concept of the present disclosure is to position the occluding device partially within the tubular organ and partially outside of the tubular organ. The occluding device is anchored in at least one position within the tubular organ, which prevents its movement in particular towards the opening of the tubular organ and the portion that is outside the tubular organ is sufficiently large in diameter to prevent its entrance into the tubular organ, thereby ensuring that the device is retained in position. The device comprises a layer that at least partially forms its external contour and engages with the walls of the tubular organ, such that it forms at least one point of complete sealing that prevents passage of certain biological matter such as an oocyte, sperm, etc. Namely, the layer, which can be a membrane, is impermeable at least for selected types of biological matter.
The device is reversibly transitionable between a collapsed state, suitable for delivery, through a semi-expanded state during the deployment of said device, and to an expanded state suitable to occlude the tubular organ. It is to be noted that the device can transition from the collapsed state to the expanded state, as well as back from the expanded state to the collapsed state, many times.
Typically, the device comprises a flexible frame that changes its shape between the collapsed state and the expanded state, and the impermeable layer is supported by said frame, at least partially. The expansion and collapsing of the frame also result in a change in the shape of the impermeable layer, which is flexible and capable of undergoing such changes.

### TECHNOLOGICAL FIELD

The present disclosure is in the field of occluding devices for tubular organs.

### BACKGROUND ART

References considered to be relevant as background to the presently disclosed subject matter are listed below:
WO 2021/245653
US 2017/0202702

Acknowledgement of the above references herein is not to be inferred as meaning that these are in any way relevant to the patentability of the presently disclosed subject matter.

### GENERAL DESCRIPTION

The present disclosure provides an occluding device, a method for deploying and retrieving it and a method for occluding a tubular organ. The concept of the present disclosure is to position the occluding device partially within the tubular organ and partially outside of the tubular organ. The occluding device is anchored in at least one position within the tubular organ, which prevents its movement in particular towards the opening of the tubular organ and the portion that is outside the tubular organ is sufficiently large in diameter to prevent its entrance into the tubular organ, thereby ensuring that the device is retained in position. The device comprises a layer that at least partially forms its external contour and engages with the walls of the tubular organ, such that it forms at least one point of complete sealing that prevents passage of certain biological matter such as an oocyte, sperm, etc. Namely, the layer, which can be a membrane, is impermeable at least for selected types of biological matter.

The device is reversibly transitionable between a collapsed state, suitable for delivery, through a semi-expanded state during the deployment of said device, and to an expanded state suitable to occlude the tubular organ. It is to be noted that the device can transition from the collapsed state to the expanded state, as well as back from the expanded state to the collapsed state, many times.

Typically, the device comprises a flexible frame that changes its shape between the collapsed state and the expanded state, and the impermeable layer is supported by said frame, at least partially. The expansion and collapsing of the frame also result in a change in the shape of the impermeable layer, which is flexible and capable of undergoing such changes.

Therefore, a first aspect of the present disclosure provides a device for occluding a flow (any directional flow) of selected types of biological matter in a tubular organ, e.g. to prevent interaction between biological materials from both sides of the lumen. The term organ should be interpreted as any organ in a living body, including tubular vessels. The device comprising a flexible frame reversibly expandable from a collapsed state to an expanded state. A membrane, namely an elastic sealing layer, is supported, at least partially, by the flexible frame and being peripheral thereto. The membrane is impermeable at least to selected types of biological matter, such as reproductive cells, blood cells, species of bacteria, etc. The flexible frame and/or the membrane define an internal volume of the device. Namely, the internal volume is defined by a volume inner to the external contour of the device that can be defined either by the membrane, the flexible frame, or any combination of portions of the membrane and the flexible frame thereof. When the device is in the expanded state, an external contour of the device defines a proximal portion dimensioned to be larger in diameter than an opening of the tubular organ to be anchored or engaged thereto and optionally sealing it. The dimension of the proximal portion ensures that the device does not migrate into the tubular organ. The external contour further defines a distal portion dimensioned to be anchored to or retained by the walls of the tubular vessel due to the pressure applied by the walls on the distal portion while it is within the tubular organ, thereby sealing off the passage of any one of said biological matter.

The device is made of biocompatible materials, including biological materials where applicable. Namely, each element separately is made of biocompatible materials.

It is to be noted that any combination of the described embodiments with respect to any aspect of this present disclosure is applicable. In other words, any aspect of the present disclosure can be defined by any combination of the described embodiments.

Furthermore, unless stated otherwise, all the embodiments of the device refer to the device in its expanded state.

In some embodiments of the device, said proximal portion has at least one portion having a larger cross-sectional dimension than the distal portion.

In some embodiments of the device, the distal portion is dimensioned to have a circumference larger than a portion of the tubular organ it is intended to be positioned in.

In some embodiments of the device, the external contour is determined by said membrane.

In some embodiments of the device, first portions of the external contour are defined by the membrane and second portions of the external contour are defined by the flexible frame.

In some embodiments of the device, the flexible frame is a braided mesh or laser cut tube mesh.

In some embodiments of the device, the flexible frame is formed of a crisscross mesh.

In some embodiments of the device, the flexible frame is composed of a biocompatible metal or alloy, such as nitinol or stainless steel or any other material.

In some embodiments of the device, the membrane is composed of biocompatible flexible elastomers, such as silicon, Teflon, ePTFE, Polyurethane or biocompatible fabric covering, such as Polyethylene terephthalate, etc.

In some embodiments of the device, the membrane is composed of biological matter, such as amniotic sac membrane, sourced from human or other mammals, e.g., of bovine or porcine origin.

In some embodiments of the device, the membrane comprises one or more openings to allow exchange of fluids between the internal volume and the surrounding of the device (i.e. lumen or tubular vessel), or the release of microbial-produced gas accumulated in said internal volume. The openings can have any form, such as cuts, slits, perforation or other profiles.

In some embodiments of the device, the membrane comprises a weak region formed at the distal portion configured to be ruptured upon exceeding a threshold pressure within the internal volume, and hence pressure applied on the weak region. This enables the transition between the expanded state and the collapsed state, typically for retrieval of the device, without experiencing internal counter pressure that may impose difficulties on the retrieval process. This may also be relevant when the perforations in the proximal portion of the device, which is designed to facilitate the exchange of fluids between the internal volume of the device and its surrounding (i.e. lumen of tubular organ) thereof, are blocked for any reason.

In some embodiments, the device further comprises a unidirectional valve formed at the distal portion of the device facilitating the discharge of fluids from the internal volume of the device to its surrounding, i.e., lumen of tubular organ. The discharge of fluids occurs only when there is a relative high pressure difference between the internal volume and the surrounding of the device.

In some embodiments of the device, a pressure sensor is disposed within the internal volume and is configured to sense the pressure within the internal volume. Upon sensing a pressure above a certain threshold, the sensor or a processing circuitry in data communication with the sensor transmits a command to open the valve and release the excess pressure. Upon sensing a reduction of the internal pressure within the internal volume below a certain threshold, an additional command is sent to close the valve.

In some embodiments of the device, the membrane encompassing the proximal portion is formed with one or more openings to facilitate the exchange of fluids between the internal volume of the device and the surrounding of the device, while the distal portion is remained sealed.

In some embodiments of the device, the device defines a longitudinal axis, the proximal portion comprises a maximal circumference in a plane normal to the longitudinal axis that is larger than a maximal circumference of the distal portion in a plane normal to the longitudinal axis, namely at parallel planes of reference.

In some embodiments of the device, the external contour defines an intermediate portion bridging or linking the proximal portion and the distal portion defining a neck portion of the device. The neck portion is intended to be positioned in the narrowest part of the tubular organ between the portion that the distal portion of the device is positioned in and the opening of the tubular organ.

In some embodiments of the device, the device defines a longitudinal axis, and said intermediate portion maintains a minimal circumference in a plane normal to the longitudinal axis that is smaller than all circumferences of both the proximal and the distal ends, defined between their two maximal circumferences in a plane normal to the longitudinal axis. In other words, the minimal circumference of the device between the maximal circumference of the distal portion and the maximal circumference of the proximal portion is comprised in the intermediate portion. This configuration is intended to fit to the geometrical dimensions of the fallopian tube, in particular the intermediate portion is intended to be positioned at the isthmus. This neck portion is intended to reduce the local pressure on the tissues, in particular in the case of the fallopian tube (owing to the arrangement of uterine layers characterizing this specific area) and transmit the stress to other device areas permitting adjustment to the cyclical muscle constriction and large, in order to prevent pain.

In some embodiments of the device, the proximal portion has a maximal diameter or a maximal circumference that is between about 20% to 400% larger than the minimal diameter or a minimal circumference of the intermediate portion.

In some embodiments of the device, the ratio between the maximal diameter or the maximal circumference of the proximal portion between the minimal diameter or the minimal circumference of the intermediate portion is between about 1.2 and 10.

In some embodiments of the device, the ratio between the maximal diameter or the maximal circumference of the distal portion between the minimal diameter or the minimal circumference of the intermediate portion is between about 1.1 and 5.

It should be noted that while the device is in the collapsed state, it is fitted to be received in a lumen of a delivering tool, such as a catheter, which has a diameter that allows its introduction into the tubular organ.

In some embodiments of the device, the proximal portion is formed with a depression, a notch, or a niche facing the proximal direction, thereby reducing the presence of the device in the cavity at the opening of the tubular organ.

In some embodiments of the device, the flexible frame comprises a protruding portion that protrudes from the depression towards the proximal direction. The device further comprising a gripping portion formed over said protruding portion for allowing gripping of the device during the deployment and the retrieval processes, e.g., for collapsing and retrieving the occluding device into a catheter.

In some embodiments of the device, the protruding portion is formed in the center of the depression and protrudes along a longitudinal axis of the device.

In some embodiments of the device, the protruding portion or the gripping portion comprises a first magnetic member configured to be magnetically coupled with a second magnetic member carried by the retrieval device. This allows to streamline the gripping process by making the association between the retrieval device and the gripping portion relatively easy.

In some embodiments of the device, the gripping portion comprises a first sleeve that fits over the protruding portion.

In some embodiments of the device, the gripping portion comprises a second sleeve that fits over the first sleeve such that a radial gap is formed between the first sleeve and the second sleeve for allowing a gripping element of a gripping device (may be a part of a delivery/retrieval device) to be received therein. For example, the gripping element can be one or more loops at the end of a delivery/retrieval device, such as a catheter.

In some embodiments of the device, a distal end of the second sleeve is cut in a profile improving the grip of the gripping element. Namely, the distal end portion is formed with a profile that can receive a gripping element, such as a loop, such that the gripping element is firmly retained there, in particular when applying a pulling manipulation on the device that results in its collapsing and retrieval into the retrieval device.

In some embodiments of the device, the distal end of the second sleeve has either one of a wavy, serrated, teeth-like or corrugated profile.

In some embodiments of the device, the protruding portion is compressible towards the distal direction, namely it is soft and flexible. This is important to prevent/minimize any risk of damage to the tissues and/or organ(s) that may engage it. For example, should the device be used to occlude the fallopian tube, the protruding portion then faces the uterine cavity, wherein the amniotic sac may engage the protruding portion during gestation. By making the protruding portion soft and flexible, the risk of damaging the amniotic sac is reduced dramatically.

In some embodiments of the device, a proximal end of the protruding portion is rounded, i.e., it does not include any sharp edges that may damage tissues or organs.

In some embodiments of the device, a proximal end of the protruding portion and/or a portion of the gripping portion are covered by a soft covering material, such as silicon, Teflon, ePTFE, Polyurethane or biocompatible fabric covering, such as Polyethylene terephthalate, etc., to prevent/minimize any risk of damage to the tissues and/or organ(s) that may engage it.

In some embodiments of the device, the depression is characterized by a longitudinal length L₁ and the protruding portion extends to a longitudinal length L₂, being shorter than L₁ such that the entire protruding portion is accommodated within the depression. The longitudinal lengths are defined parallel to the longitudinal axis of the device and in some embodiments are coaxial.

In some embodiments of the device, a maximal diameter of the depression is smaller than 5-folds than of the largest diameter of the protruding portion. In some embodiments, the maximal diameter of the depression is smaller than 4-folds, 3-folds or 2-folds than of the largest diameter of the protruding portion.

In some embodiments of the device, the proximal portion is flexible, at least towards the distal direction, to prevent/minimize any risk of damage to the tissues and/or organ(s) that may engage it.

In some embodiments of the device, the tubular organ is a fallopian tube.

In some embodiments of the device, the tubular organ is either spermatic cord or a siphon in the lung.

In some embodiments, the device is further comprising a mark visible through a delivery device for the identification of a deployment position. In some embodiments, wherein the tubular organ is a fallopian tube, the mark is intended to be identified with respect to the ostium or the opening of the fallopian tube.

In some embodiments of the device, the membrane is composed of a material inhibiting/reducing tissue growth, therefore allowing the device to be removed easily and safely without causing damage to tissues.

In some embodiments of the device, the membrane encompassing the distal portion is formed with one or more openings to facilitate the exchange of fluids between the internal volume of the device and the surrounding of the device.

In some embodiments, the device is biased to the expandable state.

In some embodiments of the device, the types of biological matters are selected from reproductive cells, blood cells, species of bacteria, inflammation liquid and liquid from hydrosalpinx region.

Yet another aspect of the present disclosure provides a kit. The kit comprising an occluding device according to any one of the above embodiments or any combination thereof. The kit further comprising a delivery device for delivering and deploying the occluding device into the tubular organ.

In some embodiments, the kit further comprising a disposable gripping device for gripping a gripping portion of the device.

In some embodiments of the kit, the gripping device comprises a second magnetic member configured to be magnetically coupled with a first magnetic member comprised in the gripping portion or proximate thereto.

Yet another aspect of the present disclosure provides a method for occluding a tubular organ. The method comprising deploying an occluding device such that (i) a distal portion of the device is anchored in the tubular organ to block the passage of selected types of biological matter, and (ii) a proximal portion of the device is positioned outside of the tubular organ. Said proximal portion is dimensioned such that it is prevented from migrating into the tubular organ through an opening of the tubular organ, hence the anchoring within the organ prevents the device from exiting the tubular organ, and the proximal portion prevents the device from migrating further into the tubular organ, so fixing it in a clinically desired position.

In some embodiments of the method, said deploying comprises expanding the occluding device from a collapsed state to an expanded state.

In some embodiments of the method, said deploying is further characterized by that (iii) an intermediate portion defining a neck portion of the device having a minimal dimension is disposed in the narrowest part of the tubular organ between the opening of the tubular organ and the portion of the tubular organ in which the distal portion is disposed in.

In some embodiments of the method, said deploying comprises ejecting the occluding device from a delivery device, such as a catheter.

In some embodiments of the method, said deploying comprises discharging the device in phases or stages, wherein each phase comprises discharging a different portion of the device and optionally withdrawing the delivery device towards the proximal direction to a certain extent, thereby maintaining the intended deployment position constant throughout the entire deployment process.

In some embodiments of the method, said ejecting is performed following positioning the delivery device such that the distal end of the delivery device is at the intended position with respect to the distal end portion of the occluding device.

In some embodiments of the method, said positioning comprises identifying that a mark on the occluding device, visible through the delivery device, reaches a desired position with respect to the tubular organ. In some embodiments, wherein the tubular organ is a fallopian tube, the mark is intended to be identified with respect to the ostium or the opening of the fallopian tube.

In some embodiments of the method, said positioning comprises identifying that a mark on a deployment device carrying the occluding device reaches a desired position with respect to the tubular organ.

In some embodiments of the method, said deploying comprises optionally retrieving the device or portions thereof into a delivery device to reposition it at a desired position within the tubular organ and discharging or ejecting the occluding device again in the new position.

In some embodiments of the method, the tubular organ is a fallopian tube.

In some embodiments of the method, the tubular organ is either spermatic cord or a siphon in the lung or any other lumen structure tubular array.

In some embodiments of the above-described device or kit, it is intended for use in a method for occluding a tubular organ.

In some embodiments of any combination of the above-described device or kit, it is intended for use in any of the above-described embodiments of the method or any combination thereof.

In some embodiments of any combination of the above-described device or kit, it is intended for use in occluding a fallopian tube suffers from hydrosalpinx. By occluding the damaged fallopian tube that has hydrosalpinx, the fertility rate of the woman may increase. In some specific cases, such as in In Vitro fertilization (IVF), fallopian tubes that suffer from hydrosalpinx may be blocked by the device of the present invention so that fluid due to hydrosalpinx will not flow into the uterus and affect the pregnancy process. Either one or both of the fallopian tubes can be occluded in such a process, according to the need.

Yet another aspect of the present disclosure provides a method for retrieving a device according to any one of the above-described embodiments or any combination thereof. The method comprising collapsing it into a collapsed state and entering it into a retrieval device.

In some embodiments of the method, collapsing comprises manipulating a gripping portion of the device.

In some embodiments, the method further comprising magnetically coupling a first magnetic member, accommodated on or proximate to the gripping portion of the device, with a second magnetic member accommodated on a retrieval device, thereby facilitating the gripping process.

### Embodiments

The following are optional embodiments and combinations thereof in accordance with aspects of the present disclosure:
1. A device for occluding a flow in a tubular organ, comprising:
   a flexible frame reversibly expandable from a collapsed state to an expanded state;
   a membrane supported, at least partially, by the flexible frame and being peripheral thereto, the membrane being impermeable at least to selected types of biological matter, the flexible frame and/or the membrane define an internal volume of the device;
   wherein in the expanded state, an external contour defines,
   a proximal portion dimensioned to be larger in diameter than an opening of the tubular organ,
   a distal portion dimensioned to be anchored within the tubular organ, for sealing off the passage of any one of said biological matter.
2. The device of embodiment 1, wherein said proximal portion has at least one portion having a larger cross-sectional dimension than the distal portion.
3. The device of embodiment 1 or 2, wherein the distal portion is dimensioned to have a circumference larger than a portion of the tubular organ it is intended to be positioned in.
4. The device of any one of embodiments 1-3, wherein the external contour is determined by said membrane.
5. The device of any one of embodiments 1-4, wherein first portions of the external contour are defined by the membrane and second portions of the external contour are defined by the flexible frame.
6. The device of any one of embodiments 1-5, wherein the flexible frame is a braided mesh.
7. The device of any one of embodiments 1-6, wherein the flexible frame is composed of a biocompatible metal or alloy.
8. The device of any one of embodiments 1-7, wherein the membrane is composed of biocompatible flexible elastomers.
9. The device of any one of embodiments 1-8, wherein the membrane comprises one or more openings to facilitate the exchange of fluids between the internal volume and the surrounding of the device.
10. The device of any one of embodiments 1-9, wherein the membrane comprises a weak region formed at the distal portion configured to be ruptured upon exceeding a threshold pressure within the internal volume.
11. The device of any one of embodiments 1-10, comprising a unidirectional valve formed at the distal portion of the device facilitating the discharge of fluids from the internal volume of the device to its surrounding.
12. The device of any one of embodiments 1-11, wherein the membrane encompassing the proximal portion is formed with one or more openings to facilitate the exchange of fluids between the internal volume of the device and the surrounding of the device.
13. The device of any one of embodiments 1-12, wherein the device defines a longitudinal axis, the proximal portion comprises a maximal circumference in a plane normal to the longitudinal axis that is larger than a maximal circumference of the distal portion in a plane normal to the longitudinal axis.
14. The device of any one of embodiments 1-13, wherein in its expanded state, the external contour defines an intermediate portion bridging the proximal portion and the distal portion defining a neck portion of the device.
15. The device of embodiment 14, wherein the device defines a longitudinal axis, and said intermediate portion maintains a minimal circumference in a plane normal to the longitudinal axis that is smaller than all circumferences of both the proximal and the distal ends defined between their two maximal circumferences in a plane normal to the longitudinal axis.
16. The device of any one of embodiments 1-15, wherein the proximal portion is formed with a depression.
17. The device of embodiment 16, wherein the flexible frame comprises a protruding portion that protrudes from the depression, the device further comprising a gripping portion formed over said protruding portion for allowing gripping of the device.
18. The device of embodiment 17, wherein the protruding portion is formed in the center of the depression and protrudes along a longitudinal axis of the device.
19. The device of embodiment 17 or 18, wherein the gripping portion comprises a first sleeve that fits over the protruding portion.
20. The device of embodiment 19, wherein the gripping portion comprises a second sleeve that fits over the first sleeve such that a gap is formed between the first sleeve and the second sleeve for allowing a gripping element of a gripping device to be received therein.
21. The device of embodiment 20, wherein a distal end of the second sleeve is cut in a profile improving the grip of the gripping element.
22. The device of embodiment 21, wherein the distal end of the second sleeve has either one of a wavy, serrated, teeth-like or corrugated profile.
23. The device of any one of embodiments 17-22, wherein the protruding portion is compressible towards the distal direction.
24. The device of any one of embodiments 17-23, wherein a proximal end of the protruding portion is rounded.
25. The device of any one of embodiments 17-24, wherein a proximal end of the protruding portion and/or a portion of the gripping portion are covered by a soft covering material.
26. The device of any one of embodiments 17-25, wherein the depression is characterized by a longitudinal length L₁ and the protruding portion extends to a longitudinal length L₂, being shorter than L₁.
27. The device of embodiment 26, wherein a maximal diameter of the depression is smaller than 5-folds than of the largest diameter of the protruding portion.
28. The device of any one of embodiments 1-27, wherein the proximal portion is flexible, at least towards the distal direction.
29. The device of any one of embodiments 1-28, wherein the tubular organ is a fallopian tube.
30. The device of any one of embodiments 1-28, wherein the tubular organ is either spermatic cord or a siphon in the lung.
31. The device of any one of embodiments 1-30, comprising a mark visible through a delivery device for identification of a deployment position.
32. The device of any one of embodiments 1-31, wherein the membrane is composed of a material inhibiting/reducing tissue growth.
33. The device of any one of embodiments 1-32, wherein the membrane encompassing the distal portion is formed with one or more openings to facilitate the exchange of fluids between the internal volume of the device and the surrounding of the device.
34. The device of any one of embodiments 1-33, being biased to the expandable state.
35. The device of any one of embodiments 1-34, wherein the types of biological matters are selected from reproductive cells, blood cells, and species of bacteria.
36. A kit comprising:
   an occluding device according to any one of embodiments 1-35;
   a delivery device for delivering and deploying the occluding device into the tubular organ.
37. The kit of embodiment 36, comprising a disposable gripping device for gripping a gripping portion of the device.
38. A method for occluding a tubular organ, comprising:
   deploying an occluding device such that (i) a distal portion of the device is anchored in the tubular organ to block the passage of selected types of biological matter, and (ii) a proximal portion of the device is positioned outside of the tubular organ, wherein said proximal portion is dimensioned such that it is prevented from migrating into the tubular organ through an opening of the tubular organ.
39. The method of embodiment 38, wherein said deploying comprises expanding the occluding device from a collapsed state to an expanded state.
40. The method of any one of embodiments 38-39, wherein said deploying is further characterized by that (iii) an intermediate portion defining a neck portion of the device having a minimal dimension is disposed in the narrowest part of the tubular organ between the opening of the tubular organ and the portion of the tubular organ in which the distal portion is disposed in.
41. The method of embodiment 40, wherein the tubular organ is a fallopian tube and the intermediate portion is disposed in the isthmus.
42. The method of any one of embodiments 38-41, wherein said deploying comprises ejecting the occluding device from a delivery device.
43. The method of embodiment 42, wherein said deploying comprises discharging the device in consecutive phases, wherein each phase comprises discharging a different portion of the device and optionally withdrawing the delivery device towards the proximal direction to a certain extent.
44. The method of any one of embodiments 38-43, wherein said ejecting is performed following positioning the delivery device such that the distal end of the delivery device is at the intended position with respect to the distal portion of the occluding device.
45. The method of embodiment 44, wherein said positioning comprises identifying that a mark on the occluding device, visible through the delivery device, reaches a desired position with respect to the tubular organ.
46. The method of embodiment 44 or 45, wherein said positioning comprises identifying that a mark on a deployment device carrying the occluding device reaches a desired position with respect to the tubular organ.
47. The method of any one of embodiments 38-46, wherein said deploying comprises optionally retrieving the device or portions thereof into a delivery device to reposition it at a desired position within the tubular organ.
48. The method of any one of embodiments 38-47, wherein the tubular organ is a fallopian tube.
49. The method of any one of embodiments 38-48, wherein the tubular organ is either spermatic cord or a siphon in the lung.
50. The device or kit of any one of embodiments 1-37, for use in a method for occluding a tubular organ.
51. The device or kit for use of embodiment 50, wherein the method is any one of embodiments 36-45.
52. The device of kit for use in occluding a fallopian tube suffers from hydrosalpinx.
53. A method for retrieving a device according to any one of embodiments 1-35, comprising collapsing it into the collapsed state and inserting it into a retrieval device.
54. The method of embodiment 53, wherein said collapsing comprises manipulating a gripping portion of the device.

### BRIEF DESCRIPTION OF THE DRAWINGS

To better understand the subject matter that is disclosed herein and to exemplify how it may be carried out in practice, embodiments will now be described, by way of non-limiting example only, with reference to the accompanying drawings, in which:

FIGS. 1A-1E are schematic illustrations of different views of a non-limiting example of the occluding device according to an aspect of the present disclosure. FIG. 1A is a side view; FIG. 1B is a longitudinal cross-sectional view of section C-C; FIG. 1C is an enlarged view of the depression and the protruding portion of the occluding device; FIG. 1D is an enlarged view of the gripping portion; and FIG. 1E is a perspective side view.

FIG. 2 is a schematic illustration of a perspective side view of a non-limiting example of a first sleeve of the gripping portion of the occluding device of FIGS. 1A-1E.

FIGS. 3A-3B are schematic illustrations of different views of a non-limiting example of the delivery/retrieval device for delivering and retrieving the occluding device of the present disclosure. FIG. 3A is a side view; FIG. 3B is a longitudinal cross-sectional view of section E-E.

FIGS. 4A-4I are schematic illustrations of different stages of the deployment of the occluding device of the present disclosure by a delivery device.

FIGS. 5A-5B are schematic illustrations of different optional gripping elements of a delivery/retrieval device; FIG. 5A shows a hook gripping element; and FIG. 5B shows a pincer gripping element.

FIGS. 6A-6B are schematic illustrations of different views of a non-limiting example of the occluding device when it engages an organ in the space of the opening of the tubular organ. FIG. 6A is a perspective view and FIG. 6B is a cross-sectional view.

FIGS. 7A-7B are schematic illustrations of another non-limiting example of an embodiment of the occluding device according to an aspect of the present disclosure. FIG. 7A is a cross-sectional view of the entire device and FIG. 7A is an enlarged cross-sectional view of the proximal portion of the occluding device.

### DETAILED DESCRIPTION

The following figures are provided to exemplify embodiments and realization of the invention of the present disclosure.

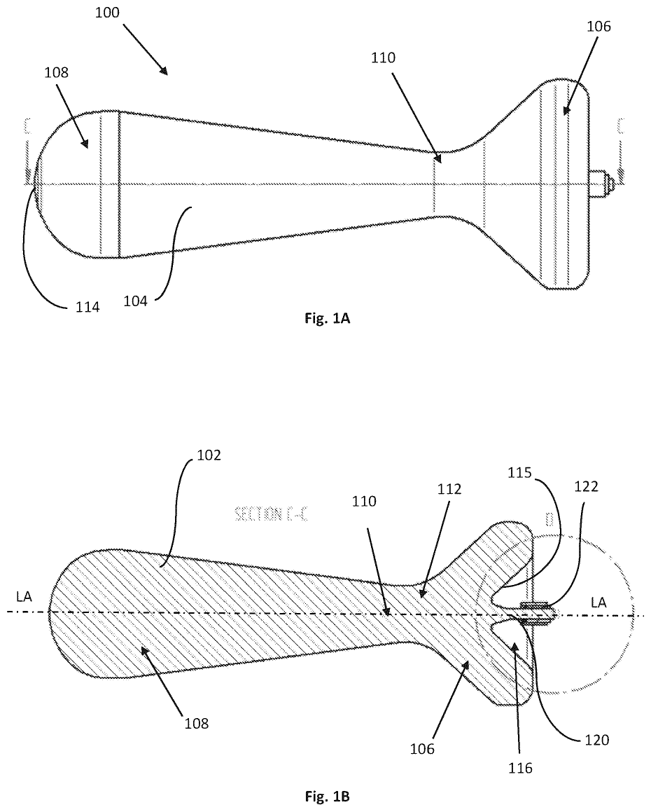

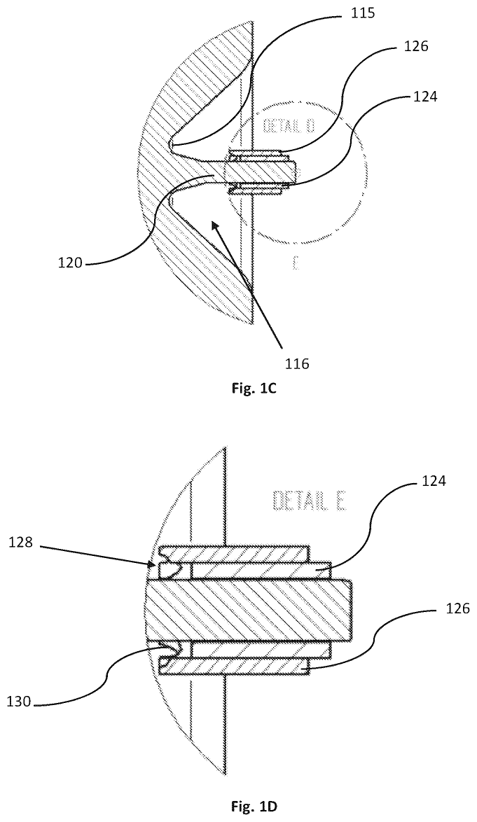

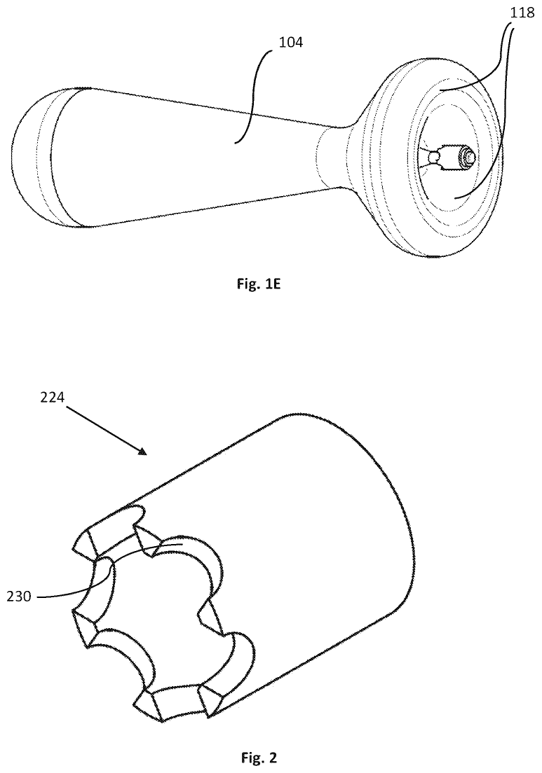

Reference is made to FIGS. 1A-1E, which are schematic illustrations of a non-limiting example of an embodiment of the occluding device according to an aspect of the present disclosure. The occluding device 100 comprises a flexible reversibly expandable frame 102 that supports, at least partially, a membrane layer 104. The frame 102 is configured to allow the transition between two states: a collapsed state, in which the device is configured to be delivered into position within the tubular organ and an expanded state, in which the device is found in its deployed state within the tubular organ. The device may also take on a semi-expanded state during the deployment procedure. The membrane 104 is a layer of material being impermeable for selected types of biological matter, such as sperm, oocyte, or any type of biological matter that is clinically desired to be blocked. The layer can be entirely impermeable to fluids or any liquids or semi-impermeable to them, namely that fluids can pass through the membrane but in a relatively very low rate.

The membrane 104 is supported by the frame 102 and forms the entire, or the vast majority, of the external contour of the device 100. The membrane 104 is flexible/elastic and is configured to change its contour between the collapsed state and the expanded state and it confines an internal empty volume of the device 102.

The device is configured to be collapsed such that it can be carried in a lumen of a catheter into the tubular organ and to be expanded into its expanded state when it is deployed in the tubular organ. FIGS. 1A-1E show the device 100 in its expanded state. In its expanded state, the device can be divided into three consecutive portions defined along a longitudinal axis LA of the device 100:

A proximal portion 106, which is intended to be positioned outside of the tubular organ and is dimensioned to be larger than the opening of the tubular organ. Thus, if a force is applied on the device that pushes it into the tubular organ, the proximal portion engages with the opening of the tubular organ and prevents its migration into the tubular organ.

A distal portion 108, which is intended to be positioned within the tubular organ and is dimensioned to be larger than the dimension of the portion of the tubular organ it is positioned in, thereby allowing it to be anchored in a desired position within the tubular organ, and preventing its migration towards the direction of the opening of the tubular organ.

An intermediate portion 110 linking the proximal portion 106 and the distal portion 108, which is intended to be positioned in the tubular organ between the most distal part where the distal portion 108 is positioned and the opening of the tubular organ. The intermediate portion 110 is a neck portion of the device 100 that is configured to be positioned in the narrowest region of the tubular organ between the opening and the most distal part.

The three portions of the device are continuous and non-discernable and are characterized by their different dimensions with respect to the external contour of the device 100. In particular, each portion is characterized by unique circumferences dimensions of the external contour in planes that are normal to the longitudinal axis LA. The proximal portion 106 comprises the largest circumference along the longitudinal axis LA to prevent its entrance into the tubular organ through the latter's opening; the distal portion 108 comprises a maximal circumference being smaller than that of the maximal circumference of the proximal portion 106 but larger than any circumference of the intermediate portion 110; and the intermediate portion comprises the minimal circumference between the maximal circumference of the proximal portion 106 and the maximal circumference of the distal portion 108, thereby defining a neck portion 112 of the device. The proximal portion 106 has a maximal diameter or a maximal circumference that is between about 20% to 400% larger than the minimal diameter or a minimal circumference of the intermediate portion 110.

The term “about” should be interpreted as a deviation of ±20% from the stated nominal value. For example, if the value is stated as “about 10”, then it should be understood to be in the range between 8-12.

The distal portion 108 comprises a weak portion 114, such that upon an increase of pressure within the internal volume of the device, this weak portion 114 is the first part of the device that is intended to be ruptured to release said pressure.

The device 100 may also be formed with openings in one or more parts of it, for example in the distal portion, the intermediate portion and/or the proximal portion to allow exchange of fluids between the internal volume of the device and its surrounding. By this, the risk of pressure building up within the internal volume of the device 100, while it is being deployed, reduces dramatically. Furthermore, when the device is transitioning from the expanded state to the collapsed state, it reduces its internal volume rapidly and by allowing a sufficient flow rate of fluid through such openings, high pressure is prevented from accumulating and the collapsing transition of the device faces/encounters no resistance.

In this non-limiting example, the openings are constituted by perforations 115 formed in the proximal portion 106.

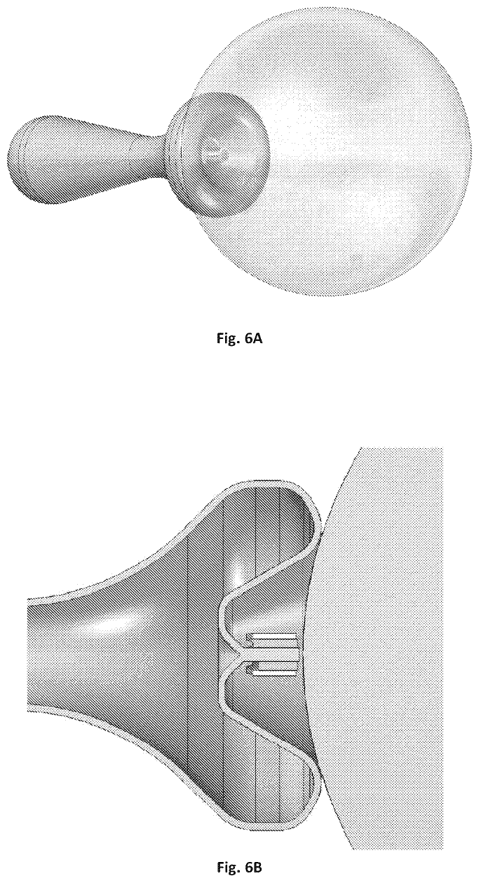

The proximal portion 106 is formed with a depression 116 or a niche, namely a central portion of the proximal face 118 of the device is depressed to some extent to maintain a minimal presence in the space immediately proximate to the opening of the tubular organ. This reduces the risk of damaging tissues and/or organs that may be present or surround the opening of the tubular organ. For example, when the tubular organ is a fallopian tube and it opens to the uterine, during pregnancy the gestational sac can grow and engage the device. Thus, the proximal end is shaped with the depression to avoid any damaging to the gestational sac, as can be seen in FIGS. 6A-6B. The perforations 115 can be formed either in a portion of the depression 116 or in other portions of the proximal portion 106. A protruding portion 120 of the frame 102 protrudes from a central portion of the depression 116 and a gripping portion 122 is formed on said protruding portion 120 for allowing a grip of a gripping element of a deploying or retrieval device. By manipulating the gripping portion 122, the device can be manipulated either to be (i) collapsed and retrieved into a retrieval device, (ii) rotated after its deployment in the tubular organ, and/or (iii) controllably deployed into the tubular organ.

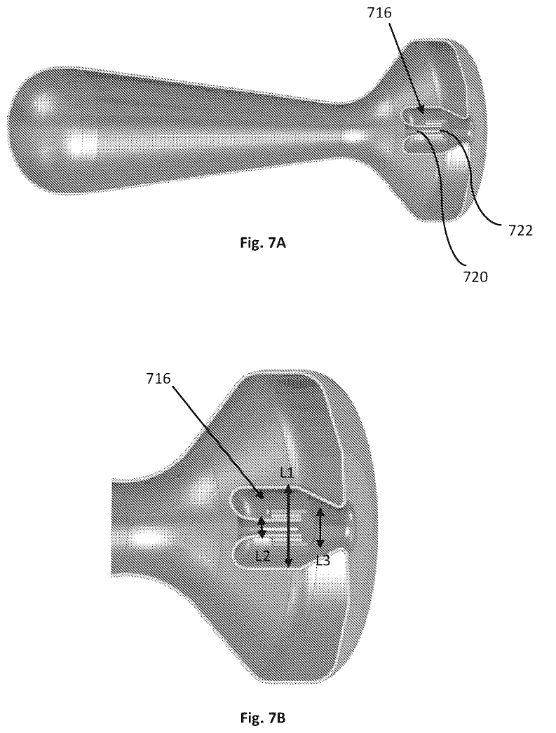

In some embodiments of the device, the protruding portion and the gripping portion are entirely accommodated within the depression, as shown in FIGS. 7A-7B. In the example in FIGS. 7A-7B, the depression 716 is relatively narrow, and its diameter L1 is in the magnitude of the diameter L2 of the protruding portion 720 and/or the diameter L3 of the gripping portion 722 (namely, it can be up to 5-folds bigger). This ensures that the protruding portion and the gripping portion will not engage an organ, such as the gestational sac.

The protruding portion 120 and the gripping portion 122 are relatively soft and flexible to prevent any damage to tissues and/or organs that may engage it. Namely, when the protruding portion 120 engages an organ, it can be bent or compressed towards the distal direction without risking the application of excessive/damaging force onto the organ. This is made possible, among other reasons, owing to the undercut design of the protruding portion and its elongated and narrow design/features.

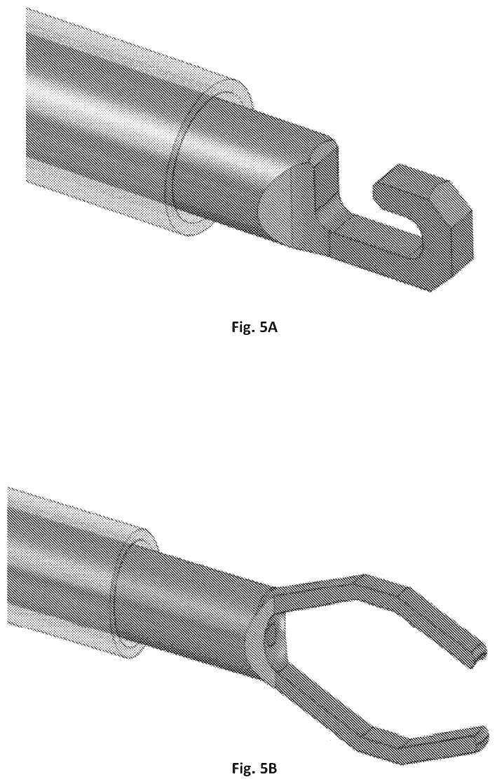

The gripping portion can be designed in several ways, each way is intended to be gripped by a different type of delivery/retrieval device. For example, the gripping portion can be a thread with a loop intended to be gripped by a hook or it can have a bulging or other grippable shape to be gripped by a pincer. Examples of gripping elements of a delivery device are shown in FIGS. 5A-5B.

In this example, the gripping portion 122 is designed to be gripped by two loops, each from a different side of the gripping portion 122. The gripping portion 122 is formed by two sleeves, a first sleeve 124 embracing the protruding portion 120 and a second sleeve 126 embracing the first sleeve 124 such that a gap that is formed between the two sleeves intended to receive the loops. The distal end 128 of the first, inner sleeve 124 is formed with cut portions 130 that provide an improved gripping mechanism, as can be best seen in FIG. 2, which is a schematic illustration showing the first sleeve 224 alone.

In the figures throughout the application, like elements of different figures were given similar reference numerals shifted by the number of hundreds corresponding to the number of the respective figure. For example, element 224 in FIG. 2 serves the same function as element 224 in FIGS. 1A-1E.

The cut portions 230 can have a crescent-like shape or any other suitable shape that can trap the loops for improving the grip mechanism.

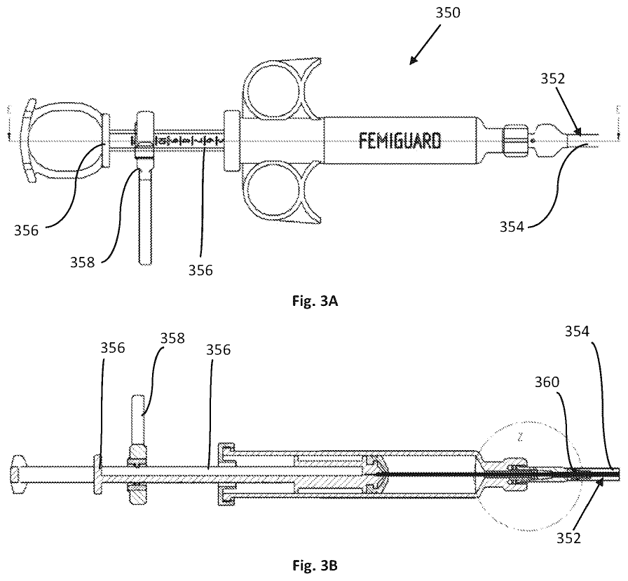

Reference is now being made to FIGS. 3A-3B, which are schematic illustrations of an example of the delivery/retrieval device (for ease of description, it is referred to throughout only as a delivery device, but it should be understood that it can also serve for retrieval) of the occluding device of the present disclosure. The delivery device 350 comprises a lumen 352 to which the occluding device is loaded to be delivered to the tubular organ. The lumen 352 is dimensioned to contain the device in its collapsed state and maintain it there until it is ejected from the delivery device 350 into the desired position of the tubular organ. Since the occluding device is biased to the expanded state, once it is ejected from the delivery device 350, it expands to its expanded state, unless there are great forces that are applied thereon, which prevent the biased transition. The delivery device is in the form of a catheter, and it includes a narrow distal edge 354 that can be guided in the body until it reaches the desired position in the tubular organ. Once reaching the desired position, by actuating a releasing mechanism 356 the occluding device starts to be discharged from the distal edge 354. In this example, the releasing mechanism 356 is in the form of a plunger that is pushed in a controllable manner, and the extent of displacement of the plunger causes a respective extend of discharge of the occluding device. The delivery device 350 comprises a safety stopping mechanism 358 that is configured to stop the displacement of the plunger 356 at a pre-set position to avoid undesired release of the occluding device. The stopping mechanism 358 can be adjusted at each stage of the deployment process according to the needs defining by/arising from that particular stage. The delivery device 350 further comprising a gripping element 360 that is designed to grip a gripping portion of the occluding device in both deployment and retrieval processes.

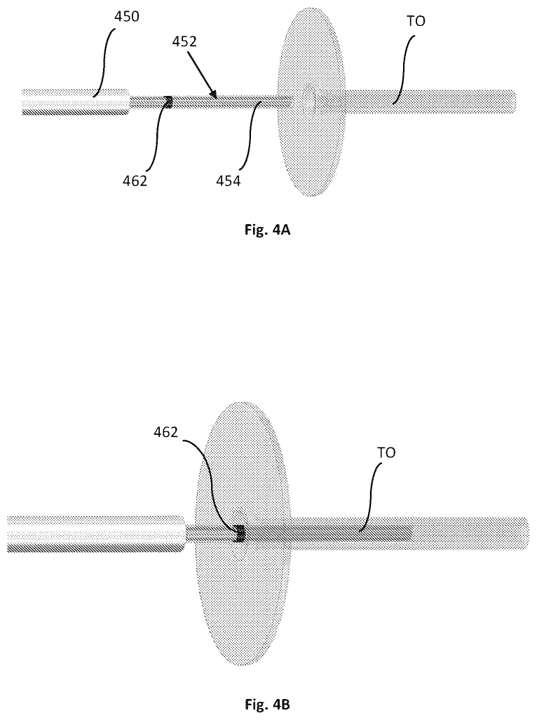

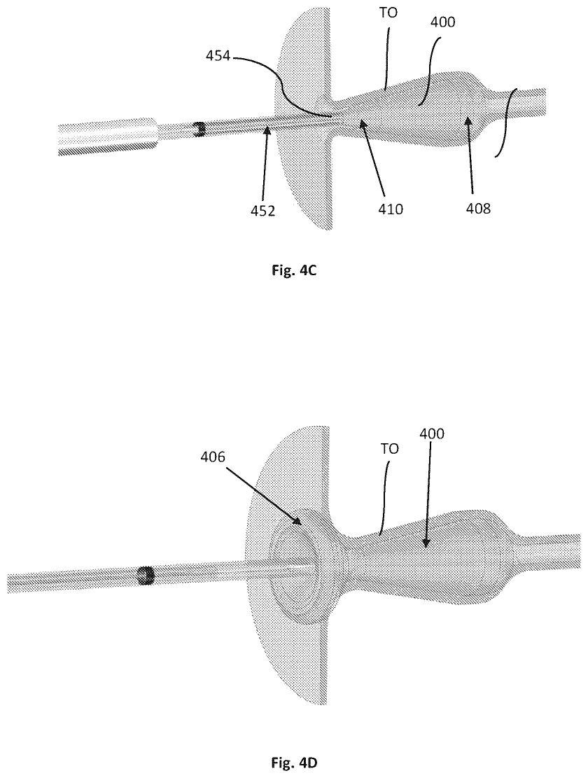

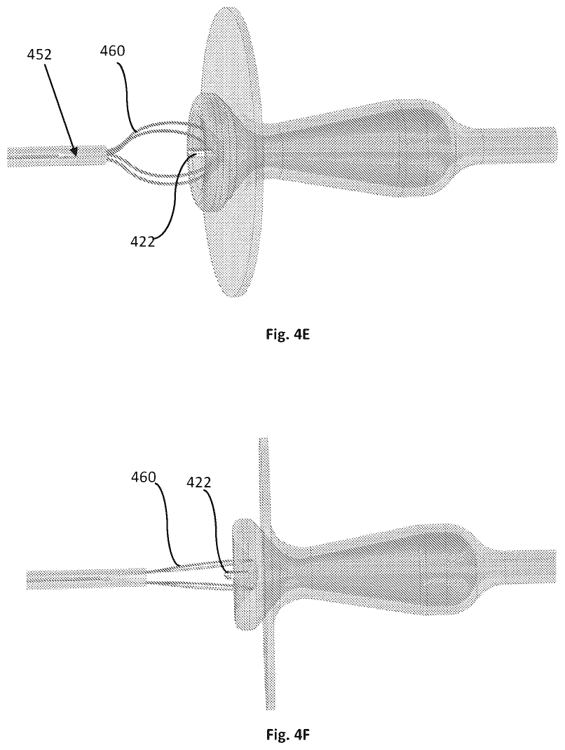

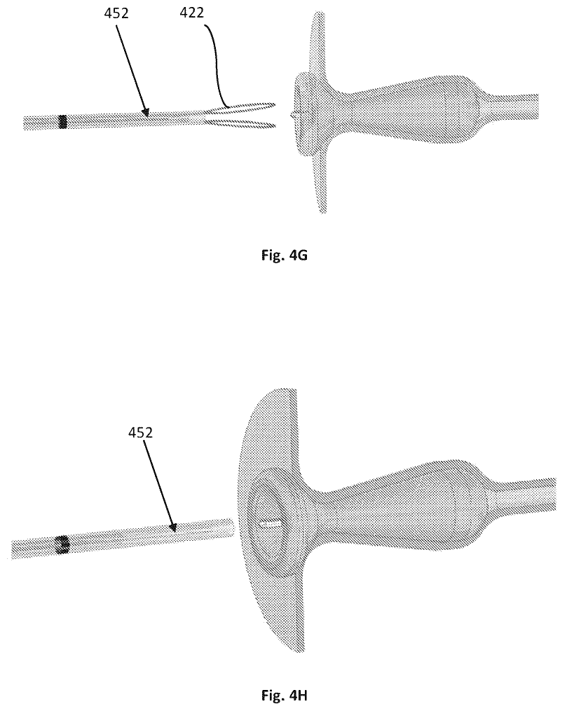

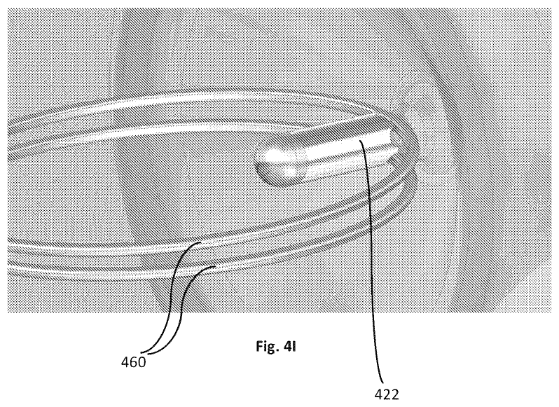

FIGS. 4A-4I show different stages of the deployment process. FIG. 4A shows the approach of the distal edge 454 to the opening of the tubular organ TO and FIG. 4B shows the distal edge 454 of the delivery device positioned within the tubular organ TO, while the occluding device 450 is still in the lumen 452 of the delivery device. The positioning can be done by identifying a position of one or more marks formed on either the delivery device, the occluding device (which can be visible through the delivery device) or a combination thereof, with respect to the tubular organ. In this example, a mark 462 is formed on the delivery device 450 that should be positioned at the opening of the tubular organ TO. FIG. 4C shows a first step of discharge of the occluding device 400 such that its distal portion 408 and its intermediate portion 410 are expanded within the tubular organ TO and the proximal portion is still confined within the lumen 452 of the delivery device. The distal edge 454 of the delivery device is pulled towards the opening of the tubular organ TO during the discharge of the occluding device and in FIG. 4C it nearly reaches the opening. In FIG. 4D, the proximal portion 406 of the occluding device 400 is discharged outside of the tubular organ TO such that it engages with the tubular organ TO, and is thereof prevented from migrating into it. FIGS. 4E-4G show the dissociation process between the gripping element 460 of the delivery device and the gripping portion 422 of the occluding device and FIG. 4H shows the retrieval process of the gripping element 460 into the lumen 452 of the delivery device 450. FIG. 4I shows an enlarged view of the association between the gripping element 460 and the gripping portion 422.

1-39. (canceled)

40. A device for occluding a flow in a tubular organ, comprising:
a flexible frame reversibly expandable from a collapsed state to an expanded state;
a membrane supported, at least partially, by the flexible frame and being peripheral thereto, the membrane being impermeable at least to selected types of biological matter, the flexible frame and/or the membrane define an internal volume of the device;
wherein in the expanded state, an external contour defines
a proximal portion dimensioned to be larger in diameter than an opening of the tubular organ,
a distal portion dimensioned to be anchored within the tubular organ, for sealing off the passage of any one of said biological matter.

41. The device of claim 40, wherein said proximal portion has at least one portion having a larger cross-sectional dimension than the distal portion.

42. The device of claim 40, wherein the distal portion is dimensioned to have a circumference larger than a portion of the tubular organ it is intended to be positioned in.

43. The device of claim 40, wherein the external contour is determined by said membrane.

44. The device of claim 40, wherein first portions of the external contour are defined by the membrane and second portions of the external contour are defined by the flexible frame.

45. The device of claim 40, wherein the membrane comprises one or more openings to facilitate the exchange of fluids between the internal volume and the surrounding of the device.

46. The device of claim 40, wherein the membrane comprises a weak region formed at the distal portion configured to be ruptured upon exceeding a threshold pressure applied thereon.

47. The device of claim 40, wherein the membrane encompassing the proximal portion is formed with one or more openings to facilitate the exchange of fluids between the internal volume of the device and the surrounding of the device.

48. The device of claim 40, wherein the device defines a longitudinal axis, the proximal portion comprises a maximal circumference in a plane normal to the longitudinal axis that is larger than a maximal circumference of the distal portion in a plane normal to the longitudinal axis.

49. The device of claim 40, wherein in its expanded state, the external contour defines an intermediate portion bridging the proximal portion and the distal portion defining a neck portion of the device;
wherein the device defines a longitudinal axis, and said intermediate portion maintains a minimal circumference in a plane normal to the longitudinal axis that is smaller than all circumferences of both the proximal and the distal ends defined between their two maximal circumferences in a plane normal to the longitudinal axis.

50. The device of claim 40, wherein the proximal portion is formed with a depression.

51. The device of claim 50, wherein the flexible frame comprises a protruding portion that protrudes from the depression, the device further comprising a gripping portion formed over said protruding portion for allowing gripping of the device.

52. The device of claim 50, wherein the gripping portion comprises a first sleeve that fits over the protruding portion.

53. The device of claim 52, wherein the gripping portion comprises a second sleeve that fits over the first sleeve such that a gap is formed between the first sleeve and the second sleeve for allowing a gripping element of a gripping device to be received therein; wherein a distal end of the second sleeve is cut in a profile improving the grip of the gripping element.

54. The device of claim 51, wherein a proximal end of the protruding portion or a portion of the gripping portion are covered by a soft covering material.

55. The device of claim 51, wherein the depression is characterized by a longitudinal length L₁ and the protruding portion extends to a longitudinal length L₂, being shorter than L₁.

56. The device of claim 40, wherein the tubular organ is any one of: a fallopian tube, spermatic cord or a siphon in the lung.

57. The device of claim 40, comprising a mark visible through a delivery device for identification of a deployment position.

58. The device of claim 40, wherein the membrane is composed of a material inhibiting/reducing tissue growth; wherein the types of biological matters are selected from reproductive cells, blood cells, species of bacteria, inflammation liquid and liquid from hydrosalpinx region.

59. A kit comprising:
an occluding device according to claim 40;
a delivery device for delivering and deploying the occluding device into the tubular organ.
